(19) United States
(12) Plant Patent Application Publication
(10) Pub. No.: US 2013/0125279 P1
(43) Pub. Date: May 16, 2013

(54) Moso bamboo plant named 'BSM-001'

(50) ;

(71) Applicant: Jackie Heinricher, Anacortes, WA (US)

(72) Inventor: Jackie Heinricher, Anacortes, WA (US)

(21) Appl. No.: 13/373,479

(22) Filed: Nov. 16, 2011

(51) Int. Cl. A01H 5/00

(52) U.S. Cl. PLT/384

(57) ABSTRACT

The present invention provides a new and distinct cultivar of Phyllostachys edulis named ‘BSM-001’, generally characterized by its rigorous growth rate and high biomass yield, compared to the control species.

### GENUS AND SPECIES

Phyllostachys edulis

### VARIETAL DENOMINATION

‘BSM-001’

### BACKGROUND

Bamboo is a group of perennial evergreens with more or less woody stems in the true grass family Poaceae, subfamily Bambusoideae, tribe Bambuseae. With over 1,250 species growing from sea level to 13,500 feet, bamboo is found on every continent except the frozen poles. Bamboo comprises a highly diverse group of plants, from tall timber varieties to groundcovers and water species. Most species are evergreen, though some may be semi-evergreen in cold winters. The American Bamboo Society lists over 225 bamboos that are cold hardy to USDA Zone 7b and below.

The uses of bamboo plants are expanding. For example, bamboo plants are being utilized for cuisine, viticulture, arboriculture, parquet flooring, laminate materials, furniture, handicrafts, textile material for paper production, structural timber, biomass production, landscape use, and benefiting environments.

A workhorse in the fight against global warming, a bamboo stand can sequester over 4 times more CO₂ than an equivalent stand of trees, simultaneously releasing 35% more oxygen. Unfortunately, like trees, bamboo has been the victim of deforestation due to population growth and increased demand for plant fiber.

There are two general patterns for the growth of bamboo: “clumping” (sympodial) and “running” (monopodial). Clumping bamboo species tend to spread slowly, as the growth pattern of the rhizomes is to simply expand the root mass gradually, similar to ornamental grasses. “Running” bamboos, on the other hand, spread mainly through their roots and/or rhizomes, which can spread widely underground and send up new culms to break through the surface. Running bamboo species are highly variable in their tendency to spread; this is related to both the species and the soil and climate conditions. Some can send out runners of several meters a year, while others can stay in the same general area for long periods.

Traditionally, bamboo has been propagated from seed as well as by root division and rhizome cuttings. Bamboo, however, does not flower and produce seeds often. Some species may not produce seed for 10 to 60 years or more, and then flower all at the same time, which is known as gregarious flowering. Certain species of bamboo are termed monocarpic due to the flowering and production of seed only once in their life cycle. Most species are not monocarpic, although they die back or become weakened after a blooming cycle and it takes a few years before the plant is completely healthy again.

Bamboo flowers are typically pollinated by wind or insects leading to cross-pollination and hybridization between different plants. The structure of the flowers prevents or discourages self-pollination, although some self-pollination can occur. Some bamboo species display selfing incompatibility. Thus, seed harvested from a bamboo plant pollinated naturally will mostly consist of a variety of genotypes from cross-pollination. Given the lengthy periodicity between flowerings, seed is not a reliable tool for production.

Phyllostachys edulis, commonly known as Moso bamboo, is the largest growing species of the genus and is considered a running bamboo. P. edulis flowers sporadically and, in general, is not considered gregarious.

Moso bamboo has commonly been used for cane production for building applications, as a large screen/hedge, for wind break, for bank stabilization and erosion control, for shoot production (e.g., as feed for panda bears), and as a specimen plant in home yards, industrial settings and botanical gardens. Moso is the most popular bamboo for manufacturing lumber products and bamboo flooring.

There is a continual need for improved cultivars of Moso bamboo, particularly for timber uses.

### SUMMARY OF THE INVENTION

The present disclosure relates to a new and distinct cultivar of Phyllostachys edulis discovered and asexually reproduced by the inventor and will be referred to hereafter by its cultivar name ‘BSM-001’. This cultivar represents a new cultivar of Phyllostachys edulis, a perennial evergreen plant in the true grass family Poaceae.

### BRIEF DESCRIPTION OF THE DRAWINGS

FIG. 1 depicts ‘BSM-001’ as compared to control Moso bamboo growing under standard in vitro culture conditions, wherein each of the plants is 5 months old and was produced via shoot induction, multiplication and division.

FIG. 2 depicts ‘BSM-001’ as compared to control Moso bamboo growing in soil under standard greenhouse conditions, wherein the plants as shown were planted into soil and placed in the greenhouse 7 months before the picture was taken.

### DETAILED BOTANICAL DESCRIPTION OF THE PLANTS

Seeds produced from the open pollination of Moso plants of unknown parentage were subsequently harvested and bulked. The resultant seeds were germinated and individual seedlings were selected for further propagation and selection. ‘BSM-001’ was selected based on its vigorous growth and its ability to produce abundant shoots in in vitro culture.

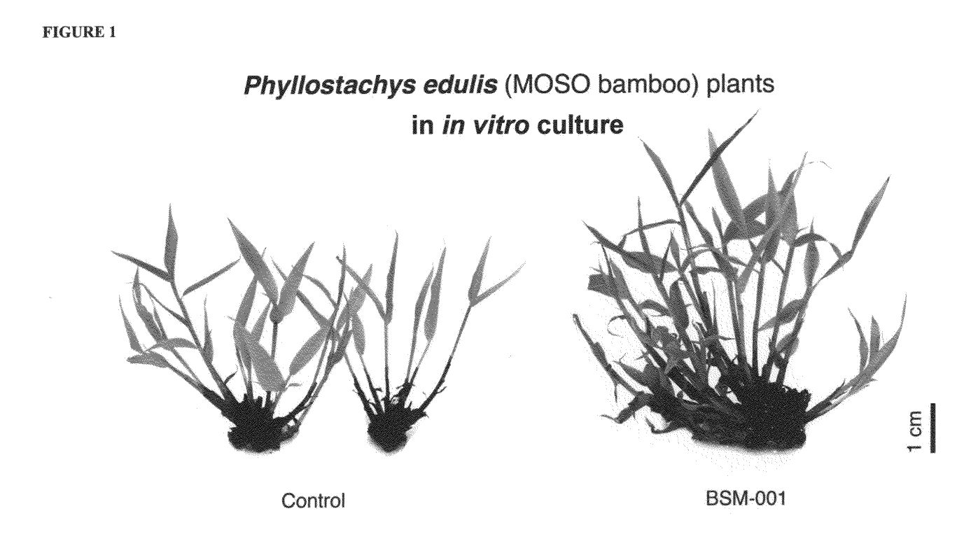

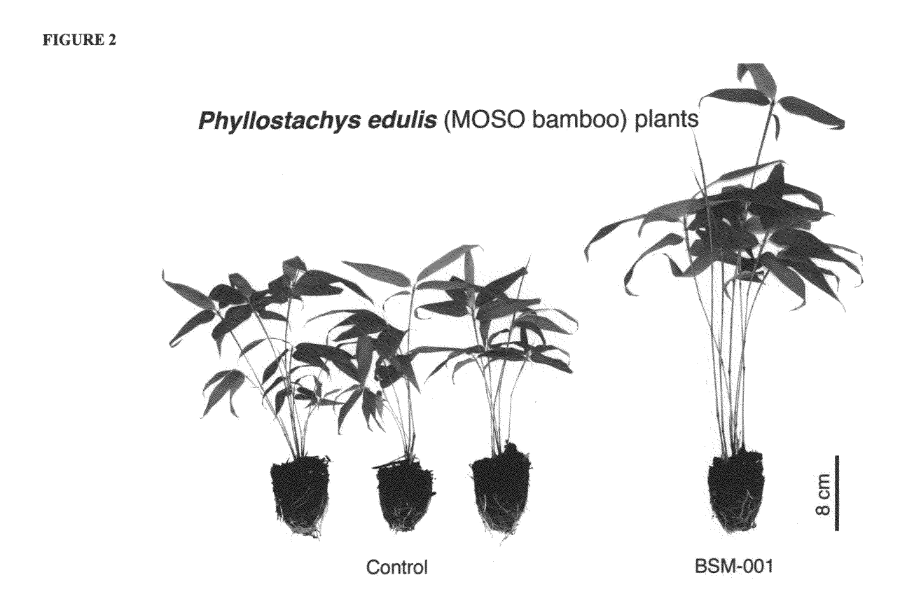

‘BSM-001’ was asexually propagated by the inventor in Mt. Vernon, Wash. using a node from a cane of ‘BSM-001’ with a lateral shoot just breaking the sheath. The resulting shoot material was micropropagated by the inventor in Mt. Vernon, Wash. on shoot induction/multiplication media for several cycles. FIG. 1 depicts multiple shoots of asexually reproduced ‘BSM-001’ after 5 months of continuous subculture cycles of 3 weeks each. The resultant asexually reproduced shoots were subsequently planted into pot soil in the greenhouse. FIG. 2 depicts a ‘BSM-001’ plant after being transferred to soil and grown in the greenhouse for 7 months. Thousands of asexually reproduced plants of ‘BSM-001’ have been produced by the inventor at Mt. Vernon, Wash. and the characteristics of this cultivar have been determined to be stable and have reproduced true to type.

The following traits have been repeatedly observed and represent the characteristics of the new cultivar. The new cultivar ‘BSM-001’ has not been observed under all possible environmental conditions. The phenotype may vary somewhat with variations in temperature, day-length, light intensity, soil types, and water and fertility levels without, however, any variance in genotype.
Botanical classification: ‘BSM-001’ is a new cultivar of Phyllostachys edulis.
Common name: Moso bamboo
Parentage: Unknown— open pollination by unknown parents.
General description:
   Propagation.—in vitro shoot culture; rhizome division; shoots from nodal sections of cane.
   Growth rate.—vigorous.
   Plant habit.—woody, tall, upright, evergreen, runner, perennial, wind tolerant.
   Plant height.—up to 70 feet.
   Stem diameter.—6-8 inches.
   Cane color.—light olive-green.
   Culms.—upright.
   Foliage.—canes are adorned with small and dainty foliage.
   Culture.—best in USDA Climate Zones 6 or 7 and warmer, preferably in the southeast portion of the U.S.
   Diseases and pests.—None known.
   Flower.—Not yet observed; Moso bamboo is an infrequent flowerer.

1. A new and distinct cultivar of Phyllostachys edulis plant named ‘BSM-001’ substantially as shown and described herein.
